(19) United States
(12) Patent Application Publication
(10) Pub. No.: US 2023/0341411 A1
(43) Pub. Date: Oct. 26, 2023

(54) METHOD AND KIT OF MEASURING CONCENTRATION OF ANALYTE

(71) Applicant: NATIONAL CHUNG CHENG UNIVERSITY, CHIA-YI (TW)

(72) Inventors: Lai-Kwan CHAU, CHIAYI CITY (TW); Chang-Yue CHIANG, TAICHUNG CITY (TW)

(21) Appl. No.: 18/214,988

(22) Filed: Jun. 27, 2023

Related U.S. Application Data
(63) Continuation-in-part of application No. 16/507,824, filed on Jul. 10, 2019.

(30) Foreign Application Priority Data
Aug. 31, 2018 (TW) 107130488

(51) Int. Cl. G01N 33/68; G01N 33/543; G01N 33/74; G01N 33/53; G01N 21/77

(52) U.S. Cl. CPC G01N 33/6848; G01N 33/54346; G01N 33/6887; G01N 33/74; G01N 33/5308; G01N 21/7703; G01N 2333/585; G01N 2333/4712; G01N 2470/04

(57) ABSTRACT

A method of measuring a concentration of an analyte is provided, including: reacting a test solution including an analyte with a nanoparticle solution including a plurality of nanoparticles and an optical waveguide element to form a sandwich-like structure; and measuring evanescent wave energy of the optical waveguide element absorbed and/or scattered by the plurality of nanoparticles after the plurality of nanoparticles forming the sandwich-like structure by using a photodetector to obtain a first signal, and calculating the concentration of the analyte based on the first signal. Wherein, a detection recognition element is conjugated on a surface of each of the plurality of nanoparticles, and a capture recognition element is conjugated on a waveguide surface of the optical waveguide element.

### CROSS-REFERENCE TO RELATED APPLICATION

This application is a continuation-in-part of U.S. patent application Ser. No. 16/507,824, filed on Jul. 10, 2019, in the U.S. Patent and Trademark Office, which claims the benefit of Taiwan Patent Application No. 107130488, filed on Aug. 31, 2018 in the Taiwan Intellectual Property Office, the disclosure of which is incorporated herein in their entirety by reference.

### BACKGROUND OF THE INVENTION

### 1. Field of the Invention

The present disclosure relates to a method and kit of measuring a concentration of an analyte, and more particularly to a method and kit of measuring a concentration of an analyte which use an optical waveguide element.

### 2. Description of the Related Art

Various types of analytical applications such as clinical diagnostics, food safety monitoring, agricultural diagnostics, metal ions detection in environmental samples, pesticide residue detection, harmful pollution substance detection, and the like, have high demands on detection sensitivity. In particular, clinical diagnosis requires high detection sensitivity and reliability for doctors to use drugs or make judgment. The small particle size of nanomaterial provides a large surface area for reaction and special properties, which is expected to provide sensing devices with higher detection sensitivity.

“Nanomaterial” is broadly defined as an ultrafine granular material composed of at least one dimension falling within a nanometer scale or a substance in the scale as a basic structural unit in a three-dimensional space. When certain particles are nanosized, say, noble metal particles, their light absorption properties are significantly changed and typically exhibit high absorbance to light in a special wavelength range. A variety of methods have been developed to detect target analytes according to the characteristics of nanoparticles having high absorbance to light at a special wavelength range, together with the conjugation of particle surface with a recognition molecule for binding with the target analyte. For example, colorimetry is a detection method based on the color change caused by the dispersion or aggregation of noble metal nanoparticles. In addition to colorimetry, a fiber-optic particle plasmon resonance sensing method also exists, which combines fiber-optic multiple total internal reflections, evanescent wave characteristics, and particle plasmon resonance properties of gold nanoparticles. This method uses noble metal nanoparticles to generate particle plasmon resonance (PPR) or localized surface plasmon resonance (LSPR) due to absorption of energy at a specific wavelength.

Although the aforementioned detection methods using nanoparticles have provided high detection sensitivity compared to conventional detection methods, there is still a need to provide a detection method with higher sensitivity. In the meantime, the accuracy of particle plasmon resonance sensing systems is greatly affected by non-specific adsorption. Therefore, there is also a demand to provide a method that is less sensitive to non-specific adsorption. Furthermore, there are unmet needs when detection results are necessary to be available on-site or achievable in short turnaround time. Then rapid on-site detection makes pivotal difference by saving time and cost and offering the opportunity to solve urgent problems. To meet the requirements of on-site detection or so-called point-of-care testing (POCT), compact, portable, easily operated, and low-cost detection devices are ideal candidates.

### SUMMARY OF THE INVENTION

Given the needs as stated above, the present disclosure aims to provide a method and kit of measuring a concentration of an analyte which has high detection sensitivity and low detection limit and is less sensitive to non-specific adsorption as well as offering desirable features such as short detection time, simple operation, and low-cost compact hardware.

According to the purpose of the present disclosure, a method for measuring a concentration of an analyte is provided, including: reacting a test solution including the analyte, a nanoparticle solution including a plurality of nanoparticles, and an optical waveguide element to form a sandwich-like structure on a waveguide surface of the optical waveguide element; and measuring evanescent wave energy of the optical waveguide element absorbed and/or scattered by the plurality of nanoparticles after the plurality of nanoparticles, the analyte, and the optical waveguide element forming the sandwich-like structure by using a photodetector to obtain a first signal, and calculating the concentration of the analyte based on the first signal. A detection recognition element is conjugated on a surface of each of the plurality of nanoparticles, and a capture recognition element is indirectly conjugated on the waveguide surface of the optical waveguide element through a second anti-nonspecific adsorption layer directly modified on the waveguide surface of the optical waveguide element. The detection recognition element and the capture recognition element are respectively bound with the analyte at different binding sites of the analyte. The term “scatter” as mentioned above refers to elastic scattering (also known as Rayleigh scattering). The analyte can be protein, peptide, deoxyribonucleic acid (DNA), cell, bacterium, virus, toxin, drug, metal ion, anion, small molecule, and so on.

Preferably, the nanoparticle may be selected from the group consisting of gold nanoparticles, silver nanoparticles, iron oxide nanoparticles, copper nanoparticles, carbon nanoparticles, cadmium selenide nanoparticles, dye-doped silicon dioxide (silica) nanoparticles, and dye-doped organic polymer nanoparticles. Preferably, the nanoparticle strongly absorb and/or scatter light in the visible-near infrared spectral region between 400 nm and 900 nm and has an extinction coefficient larger than 1×10⁷ M⁻¹ cm⁻¹ at the spectral peak. Noble metal nanoparticles, in particular, spherical gold nanoparticles larger than 5 nm in diameter and various noble metal nanoparticles with different shapes (e.g., gold nanorods, gold nanotriangle, gold nanoprisms, gold nanoshells, silver nanorods, silver nanotriangle, silver nanoprisms, etc.) have extinction coefficient larger than 1×10⁷ M⁻¹ cm⁻¹ in the region between 400 nm and 900 nm and therefore are more preferable.

Preferably, the optical waveguide element may be selected from the group consisting of a cylindrical optical waveguide element such as optical fiber, a planar optical waveguide element such as slab waveguide and channel waveguide, a tubular optical waveguide element, and a grating waveguide element.

Preferably, the detection recognition element and the capture recognition element may be each independently selected from the group consisting of antibodies, peptides, hormone receptors, lectins, saccharides, chemical recognition molecules, deoxyribonucleic acid, ribonucleic acid, and aptamers.

Preferably, a first anti-nonspecific adsorption layer may be formed between the nanoparticles and the detection recognition element.

Preferably, the step of obtaining the first signal by using the photodetector include: irradiating a single-frequency light, a narrow-band light, or a white light to a proximal end or one side of the optical waveguide element to generate the evanescent wave energy, wherein the photodetector allows the light intensity to be directly measured without spatially dispersing the light into different wavelengths by a wavelength selector. Therefore, without using a spectrometer to spatially disperse the light into different wavelengths benefits the construction of a POCT detection device with desirable features such as small size, portability, and low-cost.

Preferably, the step of obtaining the first signal by using the photodetector include: irradiating the single-frequency light, the narrow-band light, or the white light to the proximal end of the optical waveguide element and placing the photodetector at a distal end of the optical waveguide element to measure a variation of evanescent wave absorption and/or scattering when the nanoparticles approach an evanescent field of the optical waveguide element as the first signal. The variation of evanescent wave absorption and/or scattering is obtained by placing the photodetector at the distal end of the optical waveguide element to measure a variation of transmitted light intensity. Preferably, the optical waveguide element is an optical fiber or a planar waveguide element.

Preferably, the step of obtaining the first signal by using the photodetector include: irradiating the single-frequency light, the narrow-band light, or the white light to the proximal end of the optical waveguide element and placing the photodetector at a position facing the waveguide surface of the optical waveguide element to measure a variation of scattered light intensity generated by the nanoparticles approaching an evanescent field of the optical waveguide element as the first signal. Wherein, the optical waveguide element may include a plurality of sensing regions. Preferably, the optical waveguide element is an optical fiber or a planar waveguide element.

Preferably, the step of obtaining the first signal by using the photodetector include: irradiating the single-frequency light, the narrow-band light, or the white light to the one side of the optical waveguide element and placing the photodetector at a position facing the waveguide surface of the optical waveguide element to measure a variation of evanescent wave absorption and/or scattering by the nanoparticles approaching an evanescent field of the optical waveguide element as the first signal. The variation of evanescent wave absorption and/or scattering is obtained by placing the photodetector at the position facing a waveguide surface of the optical waveguide element to measure the diffracted light intensity. Wherein, the optical waveguide element may include a plurality of sensing regions. Wherein, the photodetector and the light source can be on the same side or opposite side of the waveguide surface of the optical waveguide element. Preferably, the optical waveguide element is a grating waveguide element.

Preferably, the irradiated light is a single-frequency light or a narrow-band light.

Preferably, the single-frequency or the narrow-band light is an incident light at a fixed modulation frequency.

The light detector may be selected from the group consisting of photodiodes, phototransistors, phototubes, photomultipliers, photoconductors, metal-semiconductor-metal photodetectors, charged coupled devices, and complementary metal oxide semiconductor devices.

Preferably, the second anti-nonspecific adsorption layer may include a self-assembling molecule selected from the group consisting of an alkyl silane with a carboxyl group (—COOH) or an amine group (—NH₂) at a terminal thereof, such as 11-aminoundecyltriethoxysilane (AUTES), 3-triethoxysilylpropylamine (APTES), and the like, and a self-assembling molecule selected from the group consisting of an alkyl silane with a zwitterionic group at a terminal thereof, such as sulfobetaine silane (SBSi), carboxylbetaine silane (CBSi), and phosphatidylcholine silane (PCSi); an alkyl silane with a polyethylene glycol at a terminal thereof, such as polyethylene glycol silane; and an alkyl silane with a hydroxyl group (—OH) at a terminal thereof. Preferably, the second anti-nonspecific layer may also include dextran.

Preferably, the first anti-nonspecific adsorption layer may include a self-assembling molecule selected from the group consisting of an alkyl thiol with a carboxyl group (—COOH) or an amine group (—NH₂) at a terminal thereof, and a self-assembling molecule selected from the group consisting of an alkyl thiol with a zwitterionic group at a terminal thereof, such as sulfobetaine thiol (SB-thiol), carboxylbetaine thiol (CB-thiol), phosphatidylcholine thiol (PC-thiol); an alkyl thiol with polyethylene glycol at a terminal thereof, such as polyethylene glycol thiol (PEG-thiol); and an alkyl thiol with hydroxyl group (—OH) at a terminal thereof. Preferably, the first anti-nonspecific layer may also include dextran.

According to the other purpose of the present disclosure, a kit of measuring a concentration of an analyte is provided, which includes: a light source; a nanoparticle solution including a plurality of nanoparticles and a detection recognition element being conjugated on each surface of the plurality of nanoparticles; an optical waveguide element with a capture recognition element being conjugated on a waveguide surface thereof; and a photodetector used to measure an attenuated light intensity transmitted through the optical waveguide element or the scattered light intensity or the diffracted light intensity from the optical waveguide element by the plurality of nanoparticles after the plurality of nanoparticles in the nanoparticle solution to form a sandwich-like structure on the waveguide surface of the optical waveguide element to obtain a first signal. The detection recognition element and the capture recognition element are respectively bound with the analyte at different binding sites of the analyte. The capture recognition element is indirectly conjugated on the waveguide surface of the optical waveguide element through a second anti-nonspecific adsorption layer directly modified on the waveguide surface of the optical waveguide element.

As described above, compared with a conventional optical waveguide particle plasmon resonance sensing system, the method of measuring the concentration of the analyte of the present disclosure may have one or more of the following advantages:
(1) Part (a) of FIG. 1 is a schematic diagram of a conventional optical waveguide particle plasmon resonance sensing system, wherein the nanoparticles 21 are noble metal nanoparticles. As shown in part (a) of FIG. 1, the conventional optical waveguide particle plasmon resonance sensing system quantifies by using the capture recognition element 35 on the nanoparticles 21 and a variation of the light intensity due to the change of absorption coefficient or the scattering coefficient (Aa) before and after the binding of the analyte A with the capture recognition element 35. The variation of the absorption coefficient or the scattering coefficient (Δα) is much smaller than that of the absorption coefficient or scattering coefficient (a) of the entire nanoparticle, and Δα/α is generally less than 7%. In contrast, part (c) of FIG. 1 is a schematic diagram of a sandwich-like structure formed on the waveguide surface of an optical waveguide element according to an embodiment of the present disclosure. As shown in part (c) of FIG. 1, the present disclosure quantifies by using the differences in light intensity between the zero-absorption or zero-scattering background light before forming a sandwich-like structure by the capture recognition element 35 on the waveguide surface of the optical waveguide element, the analyte A, and the detection recognition element conjugated with the nanoparticle 25, and the absorbed or scattered light after forming the sandwich-like structure. Therefore, the variation of the light intensity after absorption or scattering by the nanoparticles (proportional to a) as shown in part (c) of FIG. 1 is at least one order of magnitude greater than the variation of the light intensity of the signal (proportional to Δα) of the conventional optical waveguide particle plasmon resonance sensing system as shown in part (a) of FIG. 1. Preferably, the nanoparticles 21 as shown in part (c) of FIG. 1 are only composed of a single noble metal and do not have a core-shell structure, wherein, the core-shell structure mentioned here refers to the core-shell structure formed by one or more metal materials as well as one metal shell coated on non-metallic core, while the non-metallic anti-nonspecific adsorption material coated on the outer layer of noble metal nanoparticles is not the core-shell structure described herein.
(2) Part (b) of FIG. 1 is a schematic diagram of another conventional optical waveguide particle plasmon resonance sensing system. As shown in part (b) of FIG. 1, even if the conventional optical waveguide particle plasmon resonance sensing system is tested by using the sandwich method, which quantifies by the variation of the light intensity due to the change of absorption coefficient or the scattering coefficient (Δα+Δα′) before and after forming the sandwich-like structure by the capture recognition element 35 on the nanoparticles 21, the analyte A, and the detection recognition element 25. The variation of absorption coefficient or the scattering coefficient (Δα+Δα′) is still much smaller than the absorption coefficient or the scattering coefficient (α) of the entire nanoparticle. Thus, there is still a great difference in sensitivity improvement between the conventional system and the present disclosure.
(3) The accuracy of the conventional optical waveguide particle plasmon resonance sensing system is greatly affected by non-specific adsorption, and the present disclosure does not directly immobilize the nanoparticle on the waveguide surface of the optical waveguide element. Therefore, non-specific adsorption occurring on the waveguide surface of the optical waveguide element does not produce significant signal changes and is therefore more suitable for quantification of real and complex samples.
(4) The nanoparticles used in the present disclosure are not limited to noble metal nanoparticles and are more versatile in uses.

As described above, compared with the fluorescence or Raman scattering sensing system which uses optical waveguide combined with nanoparticle sandwich method, the method and kit of measuring the concentration of the analyte of the present disclosure still has one or more of the following advantages:
(1) The detection recognition element does not require additional labeling of fluorescent dye molecules or Raman dye molecules.
(2) The optical configuration is simpler, and cheaper optoelectronic elements can be used.
(3) Fluorescence or Raman scattering does not sufficiently utilize the multiple total internal reflection characteristics of the optical waveguide element to greatly increase the sensitivity of the measurement.

### BRIEF DESCRIPTION OF THE DRAWINGS

Parts (a) and (b) of FIG. 1 are schematic diagrams of a conventional optical waveguide particle plasmon resonance sensing system. Part (c) of FIG. 1 is a schematic diagram of a sandwich-like structure formed according to an embodiment of the present disclosure.

FIG. 2 is a flow chart of a method of measuring a concentration of an analyte according to an embodiment of the present disclosure.

FIG. 3 is a preparation schematic diagram of nanoparticles conjugated with the detection recognition element on the surface according to an embodiment of the present disclosure.

FIGS. 4A-4F are real-time detection diagrams of detecting 1×10⁻⁷, 2×10⁻⁸, 2×10⁻⁹, 2×10⁻¹⁰, 2×10⁻¹¹ and 2×10⁻¹² g/mL cTnI secondary standards according to an embodiment of the present disclosure.

FIG. 5 is a diagram of a calibration curve according to the results of FIGS. 4A-4F.

FIG. 6 is a preparation schematic diagram of an optical waveguide element modified with HS-DNA^{C} on the waveguide surface according to an embodiment of the present disclosure.

FIG. 7 is a preparation schematic diagram of nanoparticles modified with NH₂-DNA^{D} on the surface according to an embodiment of the present disclosure.

FIG. 8 is a real-time detection diagram of detecting multiple samples of silver ion secondary standards with different silver ion concentrations according to an embodiment of the present disclosure.

FIG. 9 is a diagram of a calibration curve according to the results of FIG. 8.

FIG. 10 is a real-time detection diagram of a non-specific adsorption test according to an embodiment of the present disclosure.

FIG. 11 is a real-time detection diagram of detecting multiple samples of the PCT secondary standards with different PCT concentrations according to an embodiment of the present disclosure.

FIG. 12 is a diagram of a calibration curve according to the results of FIG. 11.

FIG. 13 is a diagram of the linear correlation analysis between results obtained by using the sandwich method and the electroluminescence detection method of the present disclosure for 11 samples.

### DESCRIPTION OF THE PREFERRED EMBODIMENTS

As used herein, the term “modified” refers to be modified by physical or chemical techniques including, but not limited to, physical vapor deposition (PVD), chemical vapor deposition (CVD), electrochemical deposition, chemical reaction, self-assembly, and sol-gel process.

As used herein, the term “self-assembling molecule” refers to a specific molecule that can be closely arranged to form a self-assembled layer without the need for external force. The arrangement speed of self-assembling molecule is often affected by the solvent or the van der Waals Force of the molecule itself. In general, as the chain length of the self-assembling molecule grows longer, the hydrophobic interaction between the molecules themselves increases, thereby accelerating the alignment of the self-assembling molecules.

As used herein, “optical waveguide element” means an element including an optical waveguide, with or without a substrate for the optical waveguide, and a superstrate covering the optical waveguide as a cladding layer, wherein a portion of the cladding layer may be a sample solution. When the incident light is fully internally reflected in the optical waveguide element, the light wave travels from the optical waveguide which is a higher refractive index (RI) medium to the cladding layer which is a lower RI medium, then total internal reflection occurs and an electromagnetic wave is generated on the side of the lower RI medium, which is the evanescent wave. The amplitude of the evanescent field decays exponentially as the depth perpendicular to the interface increases. Using the optical waveguide element, the amount of change in evanescent wave absorption or scattering can be greatly increased by multiple total internal reflections, thereby greatly increasing the sensitivity of the measurement.

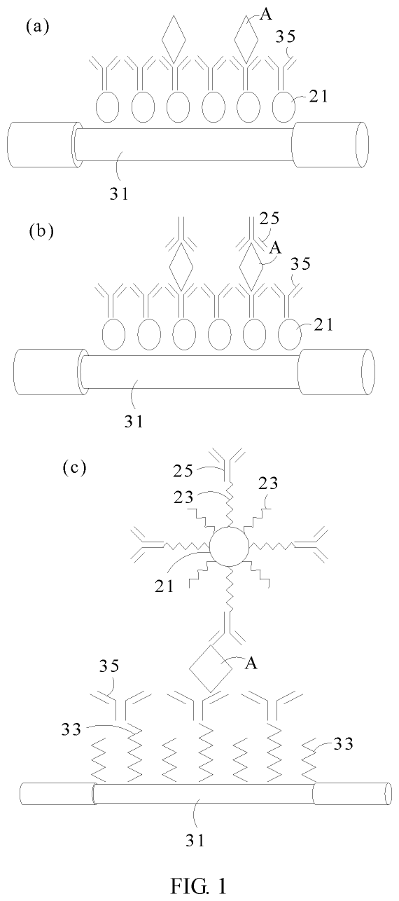

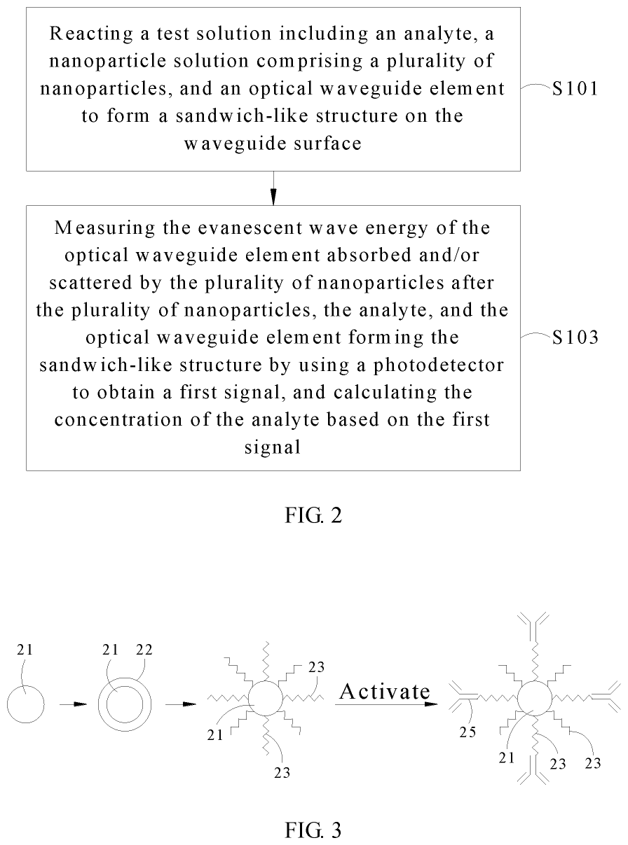

FIG. 2 is a flow chart of a method of measuring the concentration of an analyte according to an embodiment of the present disclosure. FIG. 3 is a preparation schematic diagram of nanoparticles conjugated with the detection recognition element on the surface according to an embodiment of the present disclosure. Please refer to FIG. 2. The method of measuring the concentration of the analyte according to an embodiment of the present disclosure includes step S101: reacting a test solution including an analyte, a nanoparticle solution comprising a plurality of nanoparticles, and an optical waveguide element to form a sandwich-like structure on the waveguide surface; and step S103: measuring the evanescent wave energy of the optical waveguide element absorbed and/or scattered by the plurality of nanoparticles after the plurality of nanoparticles, the analyte, and the optical waveguide element forming the sandwich-like structure by using a photodetector to obtain a first signal, and calculating the concentration of the analyte based on the first signal. Hence, the concentration of the analyte can be directly calculated based on the first signal without additional signal amplification step like silver deposition on noble metal surface and enzyme-catalyzed precipitation.

The nanoparticle solution described in step S101 is a solution including a plurality of nanoparticles whose surface conjugated with a detection recognition element. The nanoparticles whose surface conjugated with a detection recognition element may be prepared as shown in FIG. 3. Please refer to FIG. 3. Firstly, to prevent aggregation between the nanoparticles 21 and to allow the nanoparticles 21 to be stably and homogeneously distributed in an aqueous solution without the occurrence of precipitation, a temporary protective layer 22 may be modified on the surface of the nanoparticles 21, and then a self-assembling molecule having anti-nonspecific adsorption properties may form a first anti-nonspecific adsorption layer 23 on the nanoparticles 21. After the first anti-nonspecific adsorption layer 23 is activated, the detection recognition element 25 may be formed on the first anti-nonspecific adsorption layer 23, thereby forming a nanoparticle whose surface conjugated with the detection recognition element. In an embodiment, instead of forming the temporary protective layer 22, the first anti-nonspecific adsorption layer 23 may be formed directly on the nanoparticles 21. In one embodiment, the detection recognition element 25 may be also formed directly on the nanoparticles 21.

The nanoparticles 21 may be selected from one of the groups consisting of metal nanoparticles, iron oxide nanoparticles, carbon nanoparticles, cadmium selenide nanoparticles, dye-doped silica nanoparticles, and dye-doped organic polymer nanoparticles. The metal nanoparticles include gold nanoparticles, silver nanoparticles, and copper nanoparticles. Preferably, the metal nanoparticles are noble metal nanoparticles, the metal nanoparticles are only composed of a single noble metal and do not have a core-shell structure; wherein, the core-shell structure mentioned here refers to the core-shell structure formed by one or more metal materials as well as one metal shell coated on non-metallic core, while the non-metallic anti-nonspecific adsorption material coated on the outer layer of metal nanoparticles is not the core-shell structure described herein. Most preferably, the noble metal nanoparticles are gold nanoparticles. The nanoparticles may come in different shapes, such as spheres, rods, triangles, prisms, stars, and the like. The nanoparticles may be in different sizes. In an embodiment, the nanoparticles 21 are spherical with an average particle diameter from 10 to 16 nm. The nanoparticles 21 are not limited to those as mentioned above but can be any nanoparticles that adsorbed and/or scattered light in the near ultraviolet-visible-near infrared regions.

The first anti-nonspecific adsorption layer 23 may include one or more self-assembling molecules, and preferably self-assembling molecules having anti-nonspecific adsorption properties, which could be modified on the nanoparticles 21 by the self-assembly method, chemical reaction, or the sol-gel method. Examples of the alkyl thiol self-assembling molecule having anti-nonspecific adsorption characteristics may include, but are not limited to, sulfobetaine-thiol, carboxybetaine-thiol, 11-mercaptoundecyl-triethylene glycol (EG₃SH), 6-mercaptohexanol (MCH), and 2-mercaptoethanol (MCE). The first anti-nonspecific adsorption layer may include an alkyl thiol self-assembling molecule having a carboxyl group (—COOH) or an amine group (—NH₂) at a terminal thereof and one self-assembling molecule selected from the group consisting of an alkyl thiol self-assembling molecule with a zwitterionic group at a terminal thereof, such as sulfobetaine-thiol, carboxybetaine-thiol, and phospholipid choline-thiol; an alkyl thiol self-assembling molecule with the polyethylene glycol at a terminal thereof, such as polyethylene glycol thiol; and an alkyl thiol self-assembling molecule with a hydroxyl group (—OH) at a terminal thereof. In one embodiment, the first anti-nonspecific adsorption layer 23 may include an alkyl thiol self-assembling molecule with a carboxyl group (—COOH) or an amine group (—NH₂) at a terminal thereof and an alkyl thiol self-assembling molecule with a hydroxyl group (—OH) at a terminal thereof. In another embodiment, the first anti-nonspecific adsorption layer 23 may include an alkyl thiol self-assembling molecule with a carboxyl group (—COOH) or an amine group (—NH₂) at a terminal thereof and an alkyl thiol self-assembling molecule with a zwitterionic group at a terminal thereof. The alkyl thiol self-assembling molecule with a carboxyl group (—COOH) or an amine group (—NH₂) at a terminal thereof mainly provides a reaction point for the immobilization of the detection recognition element. Examples of alkyl thiol self-assembling molecule having a carboxyl group (—COOH) or an amine group (—NH₂) at a terminal thereof include, but are not limited to, 11-mercaptohexadecanoic acid (MUA), 16-mercaptohexadecanoic acid (MHDA), 11-aminoundecyltrethoxysilane (AUTES), and cystamine.

Through the alkyl thiol self-assembling molecule with a terminal carboxyl group (—COOH) or an amine group (—NH₂), after the first anti-nonspecific adsorption layer 23 is activated, the detection recognition element 25 may be indirectly conjugated on the surface of the nanoparticles 21 by an amide covalent bond (—CONH—). When the analyte interacts with the nanoparticles, the nanoparticles 21 may be sufficiently bound with the analyte through the detection recognition element 25. Therefore, according to the feature of the analyte, the detection recognition element 25 may be selected from one of the groups consisting of antibodies, peptides, hormone receptors, lectins, carbohydrates, chemical recognition molecules, deoxyribonucleic acids, ribonucleic acids, and nucleic acid aptamers.

The optical waveguide element described in step S101 is an optical waveguide element whose waveguide surface conjugated with a capture recognition element. Part (c) of FIG. 1 is a schematic diagram of a sandwich-like structure formed on the waveguide surface of the optical waveguide according to an embodiment of the present disclosure. Please refer to part (c) of FIG. 1. A second anti-nonspecific adsorption layer 33 may be formed on the waveguide surface of the optical waveguide element 31 in a similar manner to the first anti-nonspecific adsorption layer 23. The second anti-nonspecific adsorption layer 33 may include one or more self-assembling molecules, preferably self-assembling molecules having non-specific adsorption properties, which may be modified on the optical waveguide element 31 by the self-assembly method, the physical vapor deposition method, the chemical vapor deposition method, chemical reaction, or the sol-gel method. The optical waveguide element 31 may be selected from one of group consisting of a cylindrical optical waveguide element, a planar optical waveguide element, a tubular optical waveguide element, and a grating waveguide element. Preferably, the optical waveguide element 31 may be an optical fiber. Preferably, the optical fiber is a partially unclad optical fiber. Preferably, the optical waveguide element 31 may be a grating waveguide element. In an embodiment, the second anti-nonspecific adsorption layer 33 may include an alkyl silane self-assembling molecule with an amine group (—NH₂) at a terminal thereof, such as AUTES, APTES, and a self-assembling molecule selected from the group consisting of sulfobetaine silane, carboxybetaine silane, phospholipid choline silane, polyethylene glycol silane (PEG-Si), and an alkyl silane self-assembling molecule with a hydroxyl group (—OH) at a terminal thereof. In another embodiment, the second anti-nonspecific adsorption layer 33 may include dextran. The capture recognition element 35 may be indirectly conjugated on the waveguide surface of the optical waveguide element 31 by the second anti-nonspecific adsorption layer 33 in a similar manner to the detection recognition element 25. The detection recognition element 25 and the capture recognition element 35 bind with the analyte A respectively at different binding sites of the analyte. The detection recognition element 25 and the capture recognition element 35 may be the same molecule. When the analyte A comes into contact with the optical waveguide element with the waveguide surface conjugated with the capture recognition element 35 and the nanoparticles 21 with the surface conjugated with the detection recognition element 25, the detection recognition element 25 and the capture recognition element 35 bind with the analyte A at different binding sites of the analyte A, so that the optical waveguide element 31/analyte A/nanoparticles 21 sandwich-like structure is formed on the waveguide surface. The sandwich-like structure can be formed by allowing the test solution to be firstly mixed with the nanoparticle solution, making the detection recognition element 25 which is conjugated on the surface of the nanoparticles 21 sufficiently bound with the analyte A and then contacting the optical waveguide element with the capture recognition element 35 conjugated on the waveguide surface to form the sandwich-like structure on the waveguide surface. Alternatively, after the test solution with the analyte A comes into contact with the optical waveguide element 31 with the capture recognition element 35 conjugated on the waveguide surface to form the optical waveguide element 31/analyte A complex, then the nanoparticle solution including the nanoparticles 21 with the detection recognition element 25 conjugated on the surface may be added to form the optical waveguide element 31/analyte A/nanoparticles 21 sandwich-like structure.

In step S103, the photodetector may be used to measure the evanescent wave energy of the optical waveguide element 31 absorbed and/or scattered by the plurality of nanoparticles 21 of the sandwich-like structure formed in step S101 to obtain the first signal. Afterwards, the concentration of the analyte may be obtained by calculation through the first signal. Therefore, the concentration of the analyte can be directly calculated based on the first signal without additional signal amplification step like silver deposition on noble metal surface and enzyme-catalyzed precipitation. In an embodiment, through the sandwich-like structure formed in step S101, when incident light enters the proximal end of the optical waveguide element 31, the light wave undergoes multiple total internal reflections in the optical waveguide element and generates evanescent waves. The nanoparticles 21 may absorb and/or scatter the evanescent wave energy of the optical waveguide element 31. Multiple total internal reflections may greatly increase the variation of the evanescent wave energy absorbed and/or scattered, thereby massively increasing the sensitivity of the measurement. By using the photodetector, the first signal may be obtained by measuring the change of light intensity transmitted through the optical waveguide element or the scattered light intensity or the diffracted light intensity from the optical waveguide element due to the absorption and/or scattering of light by the nanoparticles 21. The concentration of the analyte may be also obtained by calculation through the first signal. In an embodiment, the transmitted light intensity (I₀) of the optical waveguide element 31 in a blank solution is first measured. Then, the transmitted light intensity (I) of the optical waveguide element 31 is measured in a sample solution. After signal processing, the transmittance (T=I/I₀) or absorbance (A=−log (I/I₀)) is obtained by calculation, which may be used for quantitative analysis of the analyte. In another embodiment, the diffracted light intensity (I₀) of the optical waveguide element 31 in a blank solution is first measured. Then, the diffracted light intensity (I) of the optical waveguide element 31 is measured in a sample solution, wherein the nanoparticles bound on the grating waveguide surface absorb and/or scatter part of the diffracted light intensity. After signal processing, the normalized intensity I/I₀ is obtained by calculation, which may be used for quantitative analysis of the analyte. In another embodiment, the scattered light intensity (I₀) of the optical waveguide element 31 in a blank solution is first measured. Then, the scattered light intensity (I) of the optical waveguide element 31 is measured in a sample solution. After signal processing, the normalized intensity (I/I₀) is obtained by calculation, which may be used for quantitative analysis of the analyte. The use of the photodetector allows the light intensity to be directly measured without spatially dispersing the light into different wavelengths by a wavelength selector. In other words, a spectrometer is not needed. Preferably, the photodetector is placed at the distal end of the optical waveguide element 31 to measure the transmitted light intensity of the nanoparticles 21. Also preferably, the photodetector is placed at a position facing the waveguide surface of the optical waveguide element 31 to measure the scattered light intensity or diffracted light intensity by the nanoparticles 21. In an embodiment, the incident light may be a single frequency light, narrow frequency band light, or white light. Preferably, the incident light may be the narrow frequency band light. In one embodiment, the device used for emitting the incident light may be a device for emitting a specific wavelength or a narrow wavelength band at a fixed modulation frequency. The signal-to-noise ratio may increase through a frequency demodulation process. For example, the device for emitting an optical signal of a specific wavelength and a fixed modulation frequency is hereinafter referred to as an optical signal output stabilization device, which includes a stable light source driving module (such as a constant voltage control module, a thermistor-based constant voltage control module, or a constant current control module), a light-emitting unit (such as light-emitting diode or laser light source), and a light source temperature stabilization module. Since the light-emitting unit is susceptible to external environment (such as temperature and airflow disturbance), which causes optical signal drifts, a passive or active light source temperature stabilization module may be used to control the light-emitting unit to further improve the optical signal stability.

The present disclosure further includes a kit (or sensing device) of the aforementioned nanoparticle solution, optical waveguide element, light source, and photodetector. The kit may be a sensing device established by the principle of Particle Plasmon Resonance (PPR). The light source may be the aforementioned optical signal output stabilization device. The sensing device may further include an optical waveguide element temperature control module and a sample injection temperature control module. The optical waveguide element temperature control module and the sample injection temperature control module are used to ensure that the injected sample temperature is consistent with the temperature of the optical waveguide element, thus increasing the reliability of the detection result.

In an embodiment, the sensing device may be selected from the group consisting of a fiber-optic particle plasmon resonance sensing device, a planar waveguide particle plasmon resonance sensing device, a tubular optical waveguide particle plasmon resonance sensing device, and a grating waveguide particle plasmon resonance sensing device.

The first signal obtained by the sensing device may be processed by a signal extracting and processing device to calculate the concentration of the analyte. Specifically, the signal extracting and processing device may include a photodetector that receives the first signal and correspondingly generates an electrical signal according to the intensity of the first signal, a current/voltage conversion circuit connected to the photodetector to convert the electrical signal into a voltage signal, and a phase-locked amplifying module that receives the voltage signal for phase-locked amplification/demodulation. The photodetector may be a photodiode detector or a phototransistor detector, and the phase-locked amplification module may be an analog phase-locked amplification module or a digital phase-locked amplification module.

Further examples are provided below to explain the disclosure in more detail.

### Embodiment 1

In embodiment 1, cardiac troponin I (cTnI) is used as an analyte, a partially unclad optical fiber is used as the optical waveguide element, and gold nanoparticles are used as nanoparticles. According to an embodiment of the method of measuring the concentration of analyte (hereinafter referred to as the sandwich method), for measuring the concentration of cTnI, it is essential to firstly respectively conjugate the detection recognition element and the capture recognition element, which may bind with cTnI at different binding sites of cTnI, on the surface of the nanoparticles and the waveguide surface of the unclad region of the optical fiber. The preparation methods of the optical waveguide element with the capture recognition element conjugated on the fiber core surface and the nanoparticles with the detection recognition element conjugated on the nanoparticle surface are described in detail below.

Preparing an Optical Waveguide Element with the Capture Recognition Element Modified on the Waveguide Surface
1. Treating the cleaned bare optical fiber with oxygen plasma for 20 minutes for preparation;
2. Formulating a solution of 5 mM AUTES and 10 mM SBSi in absolute ethanol;
3. Soaking the treated bare optical fiber in the AUTES/SBSi solution for 12 hours to allow AUTES/SBSi to be immobilized on the fiber upon self-assembled reaction;
4. Taking the modified optical fiber out of the solution, cleaning it with deionized water for several times, and then blow-drying it with nitrogen;
5. Preparing a solution of 0.08 M of diuccinimidyl suberate (DSS) in dimethyl hydrazine (DMSO);
6. Soaking the optical fiber modified by AUTES/SBSi in the DSS solution for 12 hours to activate the amine group (—NH₂) at a terminal of AUTES, so that the optical fiber may be reacted with the capture recognition element (anti-cTnI antibody, Ab^{C}) to immobilize the capture recognition element on the optical fiber;
7. Taking the modified optical fiber out of the solution, cleaning it with deionized water for several times, and then blow-drying it with nitrogen;
8. Placing the optical fiber in a microfluidic chip, using UV glue with a viscosity of 10,000 to apply to the two holes on the bottom of the chip to fix the optical fiber, and then placing the optical fiber in a UV light box for 15 minutes;
9. Injecting methanol and then deionized water into a sensing zone of the chip for cleaning to remove the UV glue vapor in the channel after the UV gel is solidified;
10. Preparing a solution of 10⁻⁴ g/mL of the capture recognition element in PBS buffer and injecting this solution into the chip for 2 hours for reaction and modification;
11. Injecting deionized water into the chip for cleaning and washing away the excess capture recognition element, so that the step of modifying the capture recognition element on the optical waveguide element is completed. If the sensing chip is not to be used immediately, this chip may be temporarily stored in a refrigerator at 4° C. for a week.

In this example, SBSi and AUTES are used to form the second anti-nonspecific adsorption layer, wherein SBSi is a zwitterionic group, which may form a layer of water molecules on the waveguide surface to resist non-specific adsorption. The terminal of AUTES is —NH₂ group, so that after activating by DSS, it reacts with the capture recognition element to conjugate the capture recognition element on the optical fiber to capture cTnI.

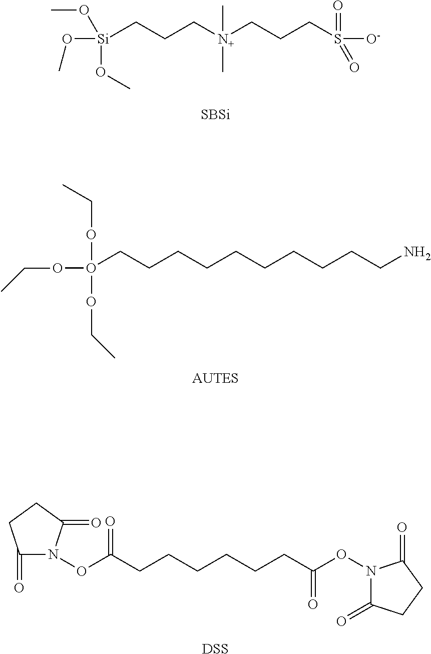

Preparing Nanoparticles with the Detection Recognition Element on the Surface
1. Preparing a solution of 2 mg/mL of Tween 20 in PBS buffer;
2. Mixing 2 mL of gold nanoparticle solution with 2 mL of Tween 20 solution and standing for 1 hour to allow Tween 20 to be uniformly coated on the gold nanoparticles, which prevents aggregation of the gold nanoparticles;
3. Preparing a solution of 0.5 mM of CB and 0.5 mM of 2-mercaptoethanol (MCE) in PBS buffer;
4. Adding 100 μL of CB-thiol/MCE solution into a gold nanoparticle solution in which the gold nanoparticles are protected by Tween 20 to allow CB-thiol/MCE to react with the gold nanoparticles for 12 hours and form a self-assembled layer on the gold nanoparticle surface;
5. Pouring the gold nanoparticle solution in which the gold nanoparticles are modified with CB-thiol/MCE into a concentrated centrifuge tube (30K) and centrifuging it at a speed of 6000 rpm for 10 minutes;
6. Discarding the supernatant and then dissolving the subnatant of the resulting gold nanoparticles back to 4 mL with a Tween 20 solution;
7. Centrifuging the solution again for 10 minutes at a speed of 6000 rpm in a concentrated centrifuge tube (30K);
8. Discarding the supernatant and dissolving the subnatant of the resulting gold nanoparticles back to 2 mL with a PBS buffer solution;
9. Formulating a solution of 1 mM of 1-ethyl-3-(dimethylaminopropyl)-carbodiimide hydrochloride (EDC) and 1 mM of N-hydroxysuccinimide (NETS) in PBS buffer;
10. Adding 2 mL of the gold nanoparticle solution in step 8 into a 100 μL solution of 1 mM EDC/NHS and reacting for 30 minutes to activate the functional group of CB-thiol/MCE;
11. Pouring the activated gold nanoparticle solution into a centrifuge tube (30K) and centrifuging it at a speed of 6000 rpm for 10 minutes;
12. Discarding the supernatant and dissolving the subnatant of the resulting gold nanoparticles back to 2 mL with a PBS buffer solution;
13. Preparing a solution of 10⁻⁵ g/mL of the detection recognition element (anti-cTnI antibody, Ab^{D}) in PBS buffer;
14. Taking 2 mL of the gold nanoparticle solution in step 12, adding a 200 μL solution of 10⁻⁵ g/mL detection recognition element, and reacting for 12 hours to conjugate the detection recognition element on the gold nanoparticles to bind with cTnI;
15. Pouring the gold nanoparticle solution in which the gold nanoparticles are conjugated with the detection recognition element into a centrifuge tube and centrifuging it at a low temperature (4° C.) for 15 minutes at a speed of 16000 rpm;
16. Discarding the supernatant and dissolving the subnatant of the resulting gold nanoparticles back to 2 mL with a PBS buffer solution, thereby obtains gold nanoparticles conjugated with the detection recognition element (hereinafter referred to as AuNP@Ab^{D}). If the gold nanoparticle solution in which the gold nanoparticles conjugated with the detection recognition element is not to be used immediately, it may be temporarily stored in a refrigerator at 4° C. for a week.

In this example, CB-thiol and MCE are used to form a first anti-nonspecific adsorption layer, wherein CB has an anti-nonspecific adsorption effect. Tween 20 is used to uniformly coat the outer layer of the gold nanoparticles to form a temporary protective layer to make the gold nanoparticles less likely to aggregate. EDC and NHS may activate the —COOH group on CB, allowing the detection recognition element with —NH₂ group to react with the —COOH group and conjugate to the gold nanoparticles.

Detecting the Concentration of cTnI Secondary Standards by the Sensing Device

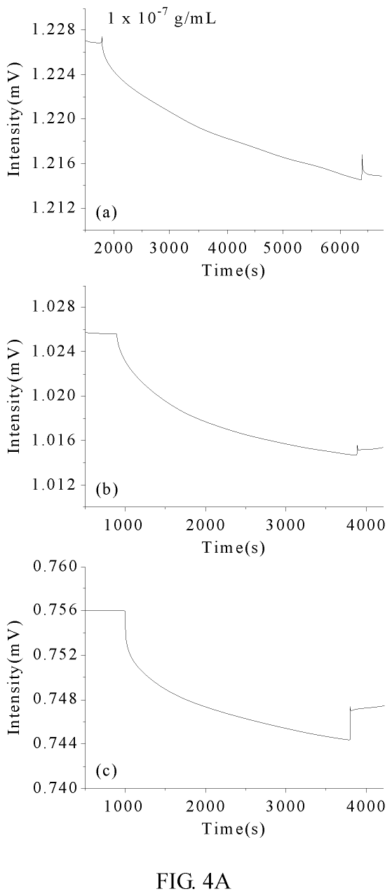

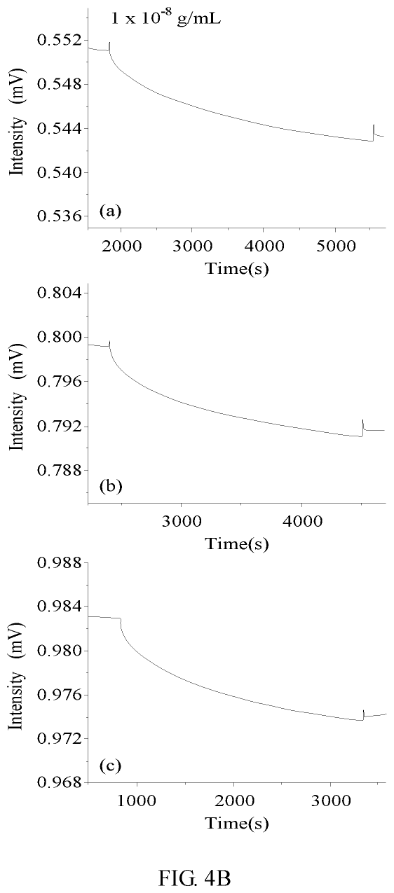

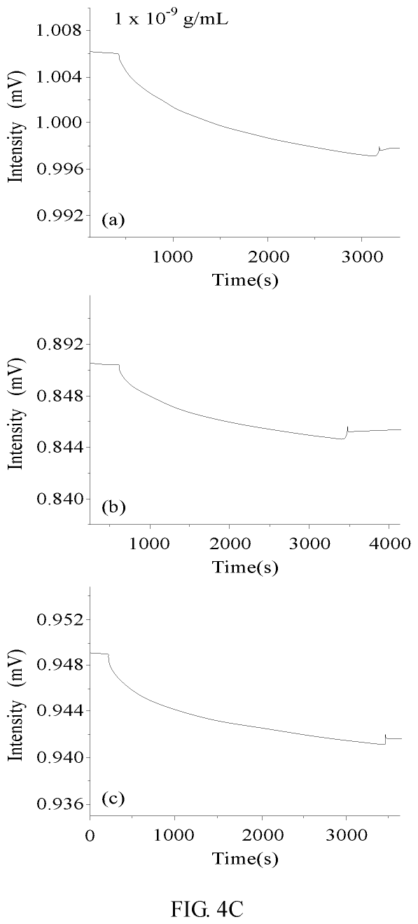

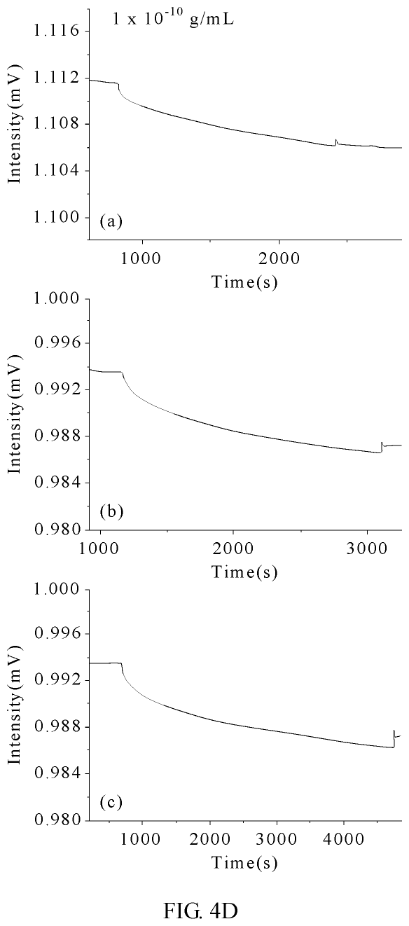

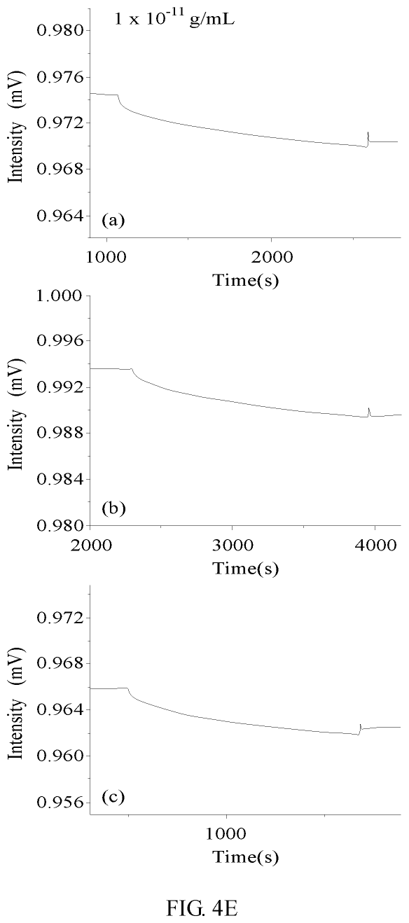

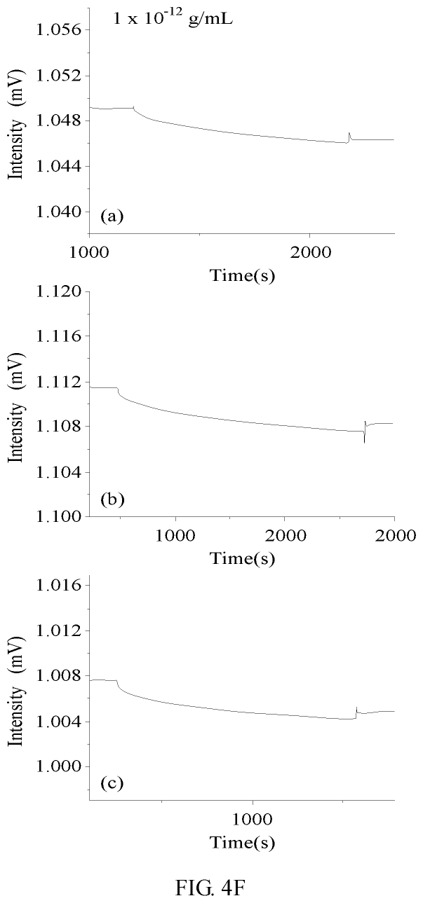

To establish a calibration curve for cTnI, a fiber-optic particle plasmon resonance sensing device is used to detect the cTnI secondary standards. The steps are described in detail as follows:
1. Preparing an optical fiber with the capture recognition element conjugated on the fiber core surface as described above;
2. Placing the optical fiber in the fiber-optic particle plasmon resonance sensing chip and injecting a PBS buffer solution into the fiber-optic particle plasmon resonance sensing chip; conducting an experiment when the baseline signal is stable;
3. Preparing the cTnI standard solutions by dissolving cTnI in PBS buffer with various concentrations (2×10⁻¹², 2×10⁻¹¹, 2×10²⁰, 2×10⁻⁹, 2×10⁻⁸, and 2×10⁻⁷ g/mL);
4. Homogeneously mixing a solution of AuNP@Ab^{D} with cTnI standard solutions of different concentrations at a volume ratio of 1 to 1 and shaking it for 15 minutes to allow the detection recognition element on the gold nanoparticles to bind with cTnI to form a secondary standard; due to dilution, the concentrations of cTnI in the secondary standards become 1×10⁻¹², 1×10⁻¹¹, 1×10⁻¹⁰, 1×10⁻⁹, 1×10⁻⁸, and 1×10⁻⁷ g/mL.
5. Making the secondary standards of different concentrations of cTnI prepared in step 4 sequentially contacted with the optical waveguide element conjugated with the capture recognition element such that the signal due to the interaction of each secondary standard with the optical waveguide element reaches a RSD of 0.008% or less in 200 seconds. Since the equilibrium time varies with concentration, a low concentration takes 15 minutes approximately, while a high concentration takes 60 minutes approximately. The results obtained are shown in FIGS. 4A-4F.
6. Establishing a calibration curve and calculate the detection limit of the sandwich method;

During the detection process, the cTnI standard is homogeneously mixed with AuNP@Ab^{D} in the first stage of the antigen-antibody binding reaction to form a secondary standard. The secondary standard is then contacted with the optical fiber conjugated with the capture recognition element. Because the specific binding interactions of the two elements, namely the detection recognition element and the capture recognition element, between the different binding sites on cTnI, the capture recognition element may bind with cTnI in the second stage of antigen-antibody binding reaction. As the reaction proceeds, the gold nanoparticles gradually approaches the evanescent field on the optical fiber to generate particle plasmon resonance and absorb the evanescent wave, while obvious signal changes may be observed on the photodetector placed at the distal end of the optical waveguide element due to the variation of the evanescent wave absorption. In another detection process, the cTnI standard may contact with the optical fiber conjugated with the capture recognition element first, and then an AuNP@Ab^{D} solution is injected into the sensing chip to allow the binding of AuNP@Ab^{D} with cTnI without the need of preparation for the secondary standards.

FIGS. 4A-4F are real-time detection diagrams of detecting 1×10⁻⁷, 2×10⁻⁸, 2×10⁻⁹, 2×10¹⁰, 2×10¹¹ and 2×10¹² g/mL cTnI secondary standards according to an embodiment of the present disclosure. Parts (a) to (c) of FIG. 4A are the results of three-time repetitions using the cTnI secondary standard with cTnI concentration of 1×10⁻⁷ g/mL. Parts (a) to (c) of FIG. 4B are the results of three-time repetitions using the cTnI secondary standard with cTnI concentration of 1×10⁻⁸ g/mL. Parts (a) to (c) of FIG. 4C are the results of three-time repetitions using the cTnI secondary standard with cTnI concentration of 1×10⁻⁹ g/mL. Parts (a) to (c) of FIG. 4D are the results of three-time repetitions using the cTnI secondary standard with cTnI concentration of 1×10⁻¹⁰ g/mL. Parts (a) to (c) of FIG. 4E are the results of three-time repetitions using the cTnI secondary standard with cTnI concentration of 1×10⁻¹¹ g/mL. Parts (a) to (c) of FIG. 4F are the results of three-time repetitions using the cTnI secondary standard with cTnI concentration of 1×10⁻¹² g/mL. As the concentration increases, more AuNP@Ab^{D}-cTnI complexes gradually approaches the fiber core surface, and the cTnI interacting with AuNP@Ab^{D} will bind with the capture recognition element in the second stage of antigen-antibody binding reaction. When the gold nanoparticles enter the evanescent field, particle plasmon resonance occurs and absorbs the evanescent waves, which results in significant signal variations. As the number of cTnI molecules in the secondary standard increases, the variation of the signal observed by the photodetector located at the distal end of the optical waveguide element 31 becomes more distinct due to the change of the evanescent wave absorption.

The signal value (I) of each concentration is subtracted from the signal value (I₀) of the blank solution to obtain delta I (ΔI), which is I₀−I. Then it is divided by the blank signal to obtain ΔI/I₀. Afterwards, all the signal differences ΔI/I₀ in the real-time detection diagram by the sandwich method to measure a single concentration of cTnI secondary standard are listed and as shown in Table 1 below.

TABLE 1

| | |
|---|---|
| cTnI | ΔI/I₀ |

| | | | | |
|---|---|---|---|---|
| 1 × 10⁻⁷ | g/mL | 0.0103 (a) | 0.0107 (b) | 0.0118 (c) |
| 1 × 10⁻⁸ | g/mL | 0.0094 (d) | 0.0088 (e) | 0.0092 (f) |
| 1 × 10⁻⁹ | g/mL | 0.0080 (g) | 0.0071 (h) | 0.0079 (i) |
| 1 × 10⁻¹⁰ | g/mL | 0.0059 (j) | 0.0065 (k) | 0.0066 (l) |
| 1 × 10⁻¹¹ | g/mL | 0.0044 (m) | 0.0040 (n) | 0.0036 (o) |
| 1 × 10⁻¹² | g/mL | 0.0028 (p) | 0.0027 (q) | 0.0025 (r) |

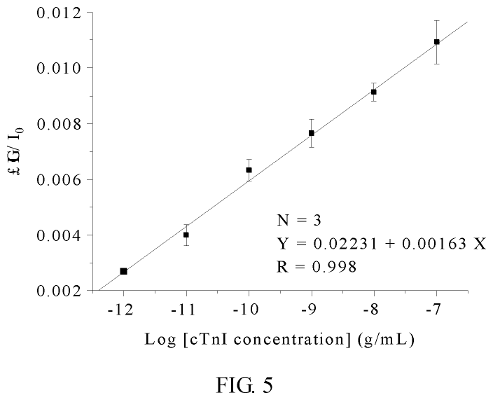

The calibration curve shown in FIG. 5 is obtained by plotting ΔI/I₀ versus log concentration of cTnI (N=3). FIG. 5 is a diagram of a calibration curve according to the result of FIGS. 4A-4F and Table 1. Please refer to FIG. 5. There is a fine linear relationship (R=0.998) in the concentration range as shown. After calculation, the detection limit for quantitative analysis of the cTnI secondary standard by the sandwich method may be obtained, which is 2.45×10⁻¹⁴ g/mL (0.0245 pg/mL, 1.02×10⁻¹⁵ M). Compared to the conventional detection method of using the fiber-optic particle plasmon resonance sensing device with only the capture recognition element having the detection limit of 1.47×10⁻⁸ g/mL, the sandwich method may increase the detection limit by nearly six orders of magnitude to provide a very low detection limit.

### Embodiment 2

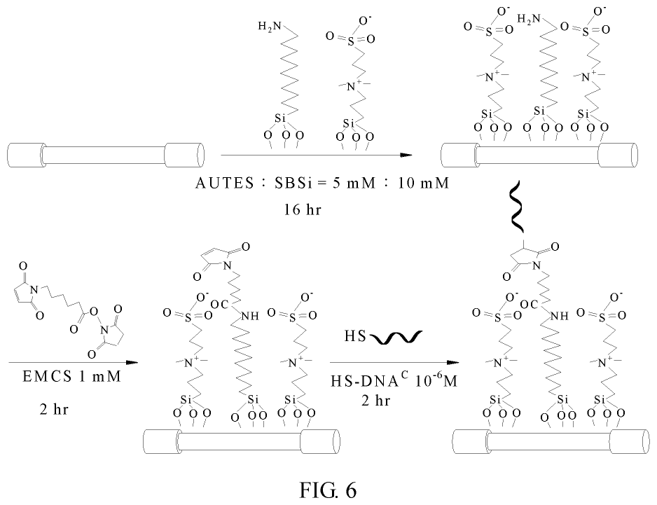

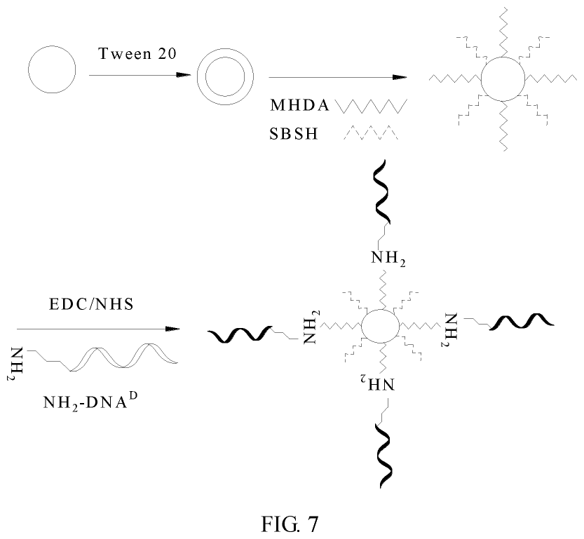

In embodiment 2, silver ions are used as the analyte, a partially unclad optical fiber is used as the optical waveguide element, and gold nanoparticles are used as the nanoparticles. According to the method for measuring the concentration of the analyte (hereinafter referred to as the sandwich method), to measure the concentration of silver ions, the detection recognition element NH₂-DNA^{D} and the capture recognition element HS-DNA, which may bind with silver ions at different positions, are respectively modified on the surface of the gold nanoparticles and the unclad region of the optical fiber surface. The preparation methods regarding the optical waveguide element with the waveguide surface modified with HS-DNA^{C} and the gold nanoparticles with the surface modified with NH₂-DNA^{D} are as respectively shown in FIG. 6 and FIG. 7.

FIG. 6 is a preparation schematic diagram of an optical waveguide element modified with HS-DNA^{C} on the waveguide surface according to an embodiment of the present disclosure. Please refer to FIG. 6. In addition to soaking the optical fiber modified with AUTES/SBSi in 1 mM solution of N-ε-malemidocaproyl-oxysuccinimide ester (EMCS) for 2 hours to activate the amine (—NH₂) group at a terminal of AUTES, and injecting a solution of 10⁻⁶ M HS-DNA^{C} into the chip for 2-hour modification, the optical waveguide element with HS-DNA^{C} modified on the waveguide surface is prepared in the same manner as in embodiment 1.

FIG. 7 is a preparation schematic diagram of nanoparticles modified with NH₂-DNA^{D} on the surface according to an embodiment of the present disclosure. Please refer to FIG. 7. The gold nanoparticles with NH₂-DNA^{D} modified on the surface are prepared by the method described in the following step:
1. Preparing a solution of 2 mg/mL of Tween 20 in PBS buffer;
2. Mixing 2 mL of gold nanoparticle solution with 2 mL of Tween 20 solution and standing for 1 hour to make Tween 20 uniformly coated on the gold nanoparticles to prevent the gold nanoparticles to aggregate;
3. Preparing a solution of 0.5 mM of MHDA and 0.5 mM of SBSH in PBS buffer;
4. Adding 100 μL of the MHDA/SBSH solution to the gold nanoparticle solution in which the gold nanoparticles are protected by Tween 20 to allow MHDA/SBSH to react overnight and form a self-assembled layer on the fiber core surface;
5. Pouring the gold nanoparticle solution in which the gold nanoparticles are modified with MHDA/SBSH into a concentrated centrifuge tube (30K) and centrifuging it at a speed of 14000 rpm for 20 minutes;
6. Discarding the supernatant and dissolving the subnatant of the resulting gold nanoparticles back to 4 mL with a Tween 20 solution;
7. Centrifuging the solution for 20 minutes at a speed of 14000 rpm in a centrifuge tube (30K);
8. Discarding the supernatant and dissolving the subnatant of the resulting gold nanoparticles back to 2 mL with a PBS buffer solution;
9. Formulating a solution of 1 mM of 1-(3-dimethylaminopropyl)-3-ethylcarbodiimide hydrochloride (EDC) and 1 mM N-hydroxysuccinimide (NETS) in PBS buffer;
10. Adding a 2 mL solution of gold nanoparticles in step 8 to a 100 μL solution of 1 mM of EDC/NHS and reacting for 10 minutes to activate the functional group of MHDA/SB SH;
11. Pouring the activated gold nanoparticle solution into a concentrated centrifuge tube (30K) and centrifuging it at 10,000 rpm for 15 minutes;
12. Discarding the supernatant and dissolving the subnatant of the resulting gold nanoparticles back to 2 mL with a PBS buffer solution;
13. Preparing a solution of 10⁻⁶ g/mL of NH₂-DNA^{D} in PBS buffer;
14. Taking a 2 mL solution of gold nanoparticles in step 12, adding a 200 μL solution of the detection recognition element with a concentration of 10⁻⁵ g/mL and reacting overnight so that NH₂-DNA^{D} may be modified on the gold nanoparticle surface to capture the silver ions;
15. Pouring the gold nanoparticle solution in which the gold nanoparticles are modified with the detection recognition element into a centrifuge tube and centrifuging it at a speed of 14000 rpm for 15 minutes at a low temperature (4° C.);
16. Discarding the supernatant and dissolving the subnatant of the resulting gold nanoparticles back to 2 mL with a PBS buffer solution, thereby obtains gold nanoparticles with NH₂-DNA^{D} modified on the surface (hereinafter referred to as AuNP@DNA^{D}). If the gold nanoparticle solution in which the gold nanoparticles are modified with NH₂-DNA^{D} is not to be used immediately, it may be temporarily stored in a refrigerator at 4° C. for one week.

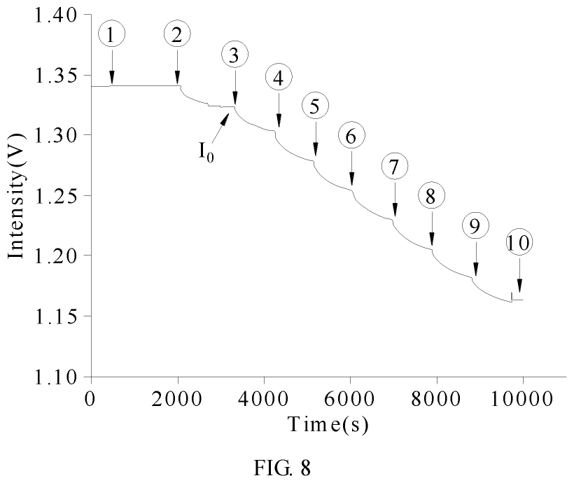

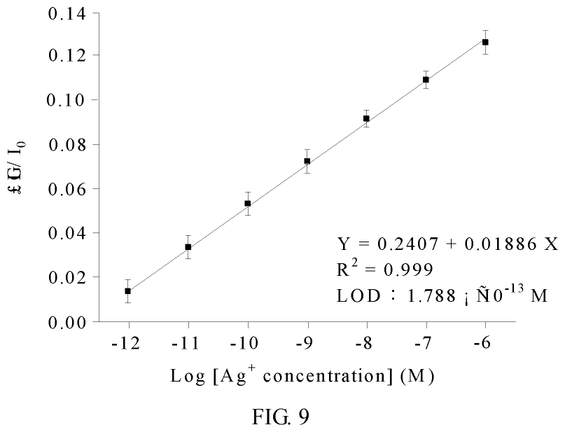

After completion of the preparation for the optical waveguide element with HS-DNA^{C} modified on the fiber core surface and gold nanoparticles with NH₂-DNA^{D} modified on the nanoparticle surface, the step of using the sensing device for determination of the concentration of the cTnI secondary standards as described in embodiment 1 is considered to establish a calibration curve for silver ions. Because HS-DNA c and NH₂-DNA^{D} contain at least a cytosine-cytosine (C-C) mismatch, they may form at least a cytosine-Ag+-cytosine (C—Ag+—C) base pair with a silver ion. The remaining base pairs are complementary to each other. Since most of them are complementary base pairs, as the reaction proceeds, the gold nanoparticles may gradually approach the fiber core surface and absorb the evanescent wave. Therefore, with the photodetector placed at the distal end of the optical waveguide element, a significant signal variation may be observed due to the change of the evanescent wave absorption. Furthermore, different signal variations come with different concentrations of silver ion standards. FIG. 8 is a real-time detection diagram of detecting multiple samples of silver ion secondary standards with increasing concentration according to an embodiment of the present disclosure. As shown in FIG. 8, the secondary standards with different silver ion concentrations are sequentially contacting one optical waveguide element modified with HS-DNA c from low silver ion concentration to high silver ion concentration (Pure water \textcircled{1}, blank \textcircled{2}, 10⁻¹² M \textcircled{3}, 10⁻¹¹ M \textcircled{4}, 10⁻¹⁰ M \textcircled{5}, 10⁻⁹ M \textcircled{6}, 10⁻⁸ M \textcircled{7}, 10⁻⁷ M \textcircled{8}, 10⁻⁶ M \textcircled{9}, pure water \textcircled{10}) with each concentration waiting for 15 minutes. FIG. 9 is a diagram of a calibration curve according to the result of FIG. 8. From FIG. 9, it is known that the embodiment of the present disclosure has a fine linearity with log silver ion concentration (R²=0.999), and the detection limit is 1.788×10⁻¹³ M.

### Embodiment 3

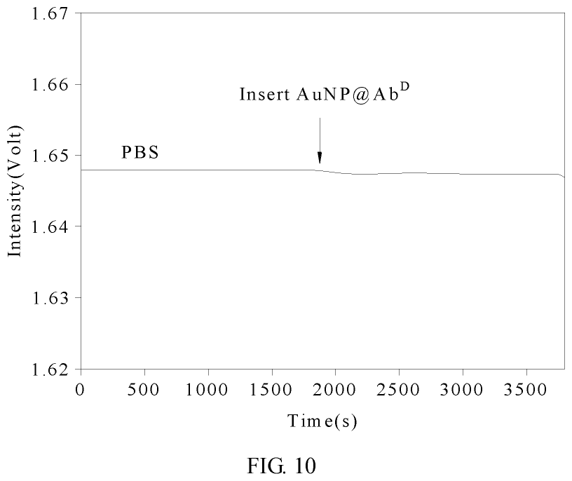

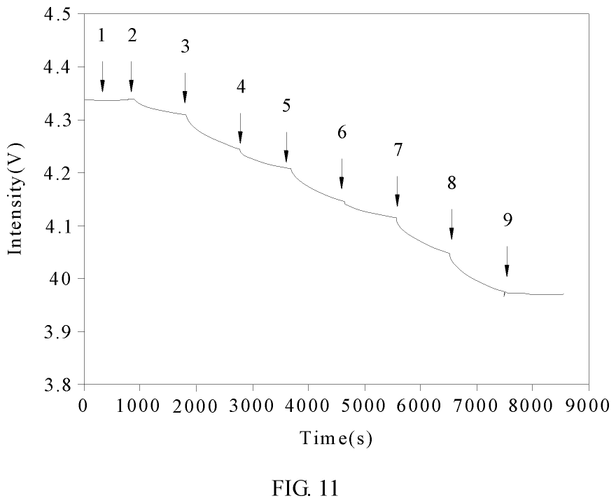

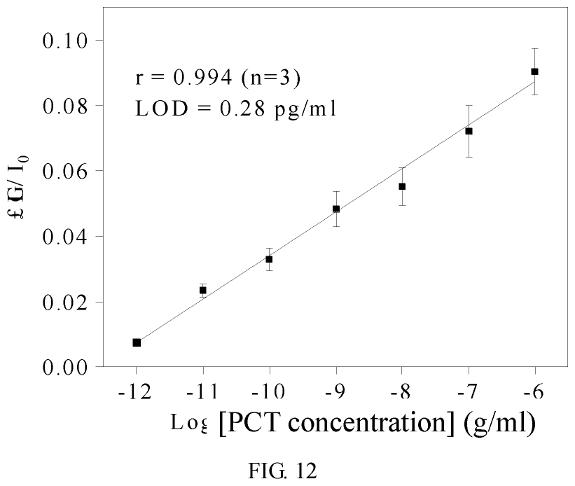

In embodiment 3, a sepsis biomarker procalcitonin (PCT) is used as the analyte, a partially unclad optical fiber is used as the optical waveguide element, and gold nanoparticles is used as the nanoparticles. To avoid the interference from other interfering biomolecules in blood samples, two types of anti-nonspecific adsorption molecules are used in embodiment 3. First, sulfobetaine silane molecules are mixed with AUTES linker molecules in a self-assembly reaction to form the second anti-nonspecific adsorption layer. Second, sulfobetaine-thiol molecules are mixed with MHDA linker molecules in a self-assembly reaction to form the first anti-nonspecific adsorption layer. A non-specific adsorption test was performed by injecting solutions of AuNP@Ab^{D} (1.0×10⁻⁷, 1.0×10⁻⁴ g/ml, where Ab^{D} is Anti-PCT^{D}) in PBS buffer. The non-specific adsorption test results are as shown in FIG. 10 according to an embodiment of the present disclosure. From FIG. 10, it can be seen that when only AuNP@Ab^{D}, but without PCT, exists in the PBS buffer solution, the signal intensity measured by the sensing device is not significantly different from the background signal. That is, the second anti-nonspecific adsorption layer may effectively prevent non-specific adsorption on the optical waveguide element. Next, in the same manner as described in embodiment 1, after activating AUTES by DSS, the capture recognition element will be conjugated on the aforementioned optical waveguide element. The nanoparticles with the detection recognition element conjugated on the nanoparticle surface used in embodiment 3 are the gold nanoparticles which are modified with MHDA/SBSH on the nanoparticle surface thereof and after activating the gold nanoparticles by using EDC/NHS, the detection recognition element will conjugate with the gold nanoparticles (hereinafter referred to as AuNP@Ab^{D}). After the solution of AuNP@Ab^{D} is mixed with the PCT standards with different PCT concentrations, a real-time detection diagram of detecting multiple samples of PCT secondary standards with different PCT concentrations is obtained in the same manner as described in embodiment 2. FIG. 11 is a real-time detection diagram of detecting multiple samples of the PCT secondary standards with different PCT concentrations according to an embodiment of the present disclosure, which is obtained by sequentially contacting the optical waveguide element conjugated with a capture recognition element from low PCT concentration to high PCT concentration (PBS (1), 10⁻¹² g/ml (2), 10⁻¹¹ g/ml (3), 10⁻¹⁰ g/ml (4), 10⁻⁹ g/ml (5), 10⁻⁸ g/ml (6), 10⁻⁷ g/ml (7), 10⁻⁶ g/ml (8), PBS (9)). FIG. 12 is a diagram of a calibration curve according to the result of FIG. 11.

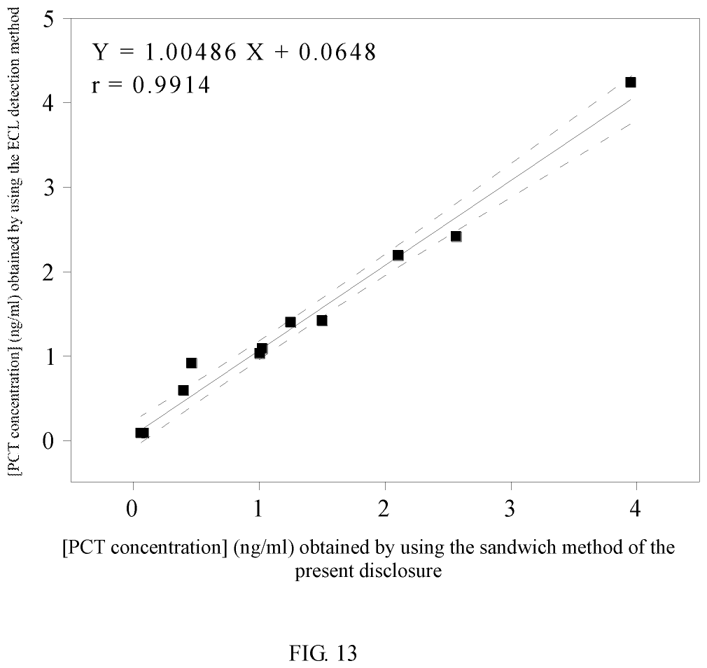

From FIGS. 11 and 12, it can be seen that, for PCT, the sandwich method of the present disclosure provides a broad linear response range from 1 pg/mL to 100 ng/mL and an extremely low detection limit of 0.28 pg/mL (0.021 pM), which is much lower than the detection limits of the detection methods using electrochemiluminescence (3.40 pM) and electrochemical detection (0.5 pM). Next, 11 blood plasma samples are separately detected by the sandwich method and the electrochemiluminescence detection method of the present disclosure, and the obtained results are analyzed using the linear correlation analysis. FIG. 13 is a diagram of the linear correlation analysis between the results obtained by using the sandwich method and the ECL detection method of the present disclosure for the 11 samples. From FIG. 13, it can be revealed that the results obtained by the two detection methods are not significantly different according to a statistical test.

The above examples have confirmed that the method of measuring the concentration of the analyte in the present disclosure may provide high detection sensitivity and low detection limit. Therefore, the method of measuring the concentration of the analyte according to the present disclosure may be used to detect the concentration of the analyte in a sample where the concentration of the analyte is too low to be measured by a conventional method. Thereby the method may meet the needs for high detection sensitivity and low detection limit of various applications such as clinical diagnostics, food safety monitoring, agricultural diagnostics, detection of metal ions in environmental samples, analysis of pesticide residues, and harmful pollutants detection.

The present disclosure has been described with some preferred embodiment thereof and it is understood that many changes and modifications in the described embodiment can be carried out without departing from the scope and the spirit of the disclosure that is intended to be limited only by the appended claims.

What is claimed is:

1. A method of measuring a concentration of an analyte without a spectrometer, comprising:
reacting a test solution comprising the analyte with a nanoparticle solution comprising a plurality of nanoparticles and an optical waveguide element to form a sandwich-like structure; and
obtaining a first signal by using a photodetector to directly measure evanescent wave energy of the optical waveguide element after evanescent waves generated in the optical waveguide element are absorbed and/or scattered by the plurality of nanoparticles of the sandwich-like structure formed of the plurality of nanoparticles, the analyte, and the optical waveguide element, and calculating the concentration of the analyte based on the first signal;
wherein a detection recognition element is indirectly conjugated on a surface of each of the plurality of nanoparticles through a first anti-nonspecific adsorption layer directly modified on the plurality of nanoparticles, and a capture recognition element is indirectly conjugated on a waveguide surface of the optical waveguide element through a second anti-nonspecific adsorption layer directly modified on the waveguide surface of the optical waveguide element;
wherein the detection recognition element and the capture recognition element are respectively bound with the analyte at different binding sites of the analyte;
wherein the plurality of nanoparticles are noble metal nanoparticles;
wherein the concentration of the analyte can be directly calculated based on the first signal without additional signal amplification step.

2. The method of measuring the concentration of the analyte according to claim 1, wherein the plurality of nanoparticles are only composed of a single noble metal and do not have a core-shell structure.

3. The method of measuring the concentration of the analyte according to claim 1, wherein the optical waveguide element is selected from the group consisting of a cylindrical optical waveguide element, a planar optical waveguide element, a tubular optical waveguide element, and a grating waveguide element.

4. The method of measuring the concentration of the analyte according to claim 1, wherein the detection recognition element and the capture recognition element are each independently selected from the group consisting of antibodies, peptides, hormone receptors, lectins, saccharides, chemical recognition molecules, deoxyribonucleic acid, ribonucleic acid, and aptamers.

5. The method of measuring the concentration of the analyte according to claim 1, wherein the first anti-nonspecific adsorption layer comprises: a first alkyl thiol self-assembling molecule with a carboxyl group (—COOH) or an amine group (—NH2) at a terminal thereof, and a second alkyl thiol self-assembling molecule selected from the group consisting of an alkyl thiol self-assembling molecule with a zwitterionic group at a terminal thereof, an alkyl thiol self-assembling molecule with a polyethylene glycol at a terminal thereof, and an alkyl thiol self-assembling molecule with a hydroxyl group (—OH) at a terminal thereof.

6. The method of measuring the concentration of the analyte according to claim 1, wherein the first anti-nonspecific adsorption layer comprises dextran.

7. The method of measuring the concentration of the analyte according to claim 1, wherein the step of obtaining the first signal by using the photodetector comprises: irradiating a single-frequency light, a narrow-band light, or a white light to a proximal end or one side of the optical waveguide element to generate the evanescent wave energy.

8. The method of measuring the concentration of the analyte according to claim 7, wherein the single-frequency or the narrow-band light is an incident light having a fixed modulation frequency.

9. The method of measuring the concentration of the analyte according to claim 7, wherein the step of obtaining the first signal by using the photodetector comprises: irradiating the single-frequency light, the narrow-band light, or the white light to the proximal end of the optical waveguide element and placing the photodetector at a distal end of the optical waveguide element to measure the evanescent wave energy corresponding to a variation of transmitted light intensity when the plurality of nanoparticles approach an evanescent field of the optical waveguide element as the first signal.

10. The method of measuring the concentration of the analyte according to claim 7, wherein the step of obtaining the first signal by using the photodetector comprises: irradiating the single-frequency light, the narrow-band light, or the white light to the proximal end of the optical waveguide element and placing the photodetector at a position facing the waveguide surface of the optical waveguide element to measure the evanescent wave energy corresponding to a variation of scattered light intensity when the plurality of nanoparticles approach an evanescent field of the optical waveguide element as the first signal.

11. The method of measuring the concentration of the analyte according to claim 10, wherein the optical waveguide element comprises a plurality of sensing regions.

12. The method of measuring the concentration of the analyte according to claim 7, wherein the step of obtaining the first signal by using the photodetector comprises: irradiating the single-frequency light, the narrow-band light, or the white light to the one side of the optical waveguide element and placing the photodetector at a position facing the waveguide surface of the optical waveguide element to measure the evanescent wave energy corresponding to a variation of diffracted light intensity when the plurality of nanoparticles approach an evanescent field of the optical waveguide element as the first signal, wherein the photodetector and the light source can be on the same side or opposite side of the waveguide surface of the optical waveguide element.

13. The method of measuring the concentration of the analyte according to claim 12, wherein the optical waveguide element is a grating waveguide element.

14. The method of measuring the concentration of the analyte according to claim 13, wherein the grating waveguide element comprises a plurality of sensing regions.

15. The method of measuring the concentration of the analyte according to claim 1, wherein the photodetector is selected from the group consisting of photodiodes, phototransistors, phototubes, photomultipliers, photoconductors, metal-semiconductor-metal photodetectors, charged coupled devices, and complementary metal oxide semiconductor devices.

16. The method of measuring the concentration of the analyte according to claim 1, wherein the second anti-nonspecific adsorption layer comprises: a first alkyl silane self-assembling molecule with a carboxyl group (—COOH) or an amine group (—NH2) at a terminal thereof, and a second alkyl silane self-assembling molecule selected from the group consisting of an alkyl silane self-assembling molecule with a zwitterionic group at a terminal thereof, an alkyl silane self-assembling molecule with a polyethylene glycol at a terminal thereof, and an alkyl silane self-assembling molecule with a hydroxyl group (—OH) at a terminal thereof.

17. The method of measuring the concentration of the analyte according to claim 1, wherein the second anti-nonspecific adsorption layer comprises dextran.

18. A kit of measuring a concentration of an analyte without a spectrometer, comprising:
a light source;
a nanoparticle solution comprising a plurality of nanoparticles and a detection recognition element being conjugated on a surface of each of the plurality of nanoparticles;
an optical waveguide element with a capture recognition element being conjugated on a waveguide surface thereof; and
a photodetector used to obtain a first signal by directly measuring evanescent wave energy of the optical waveguide element after evanescent waves generated in the optical waveguide element are absorbed and/or scattered by the plurality of nanoparticles of a sandwich-like structure formed of the plurality of nanoparticles, the analyte and the optical waveguide element;
wherein the detection recognition element and the capture recognition element are respectively bound with the analyte at different binding sites of the analyte; and
the capture recognition element is indirectly conjugated on the waveguide surface of the optical waveguide element through a second anti-nonspecific adsorption layer directly modified on the waveguide surface of the optical waveguide element;
wherein the plurality of nanoparticles are noble metal nanoparticles, the plurality of nanoparticles are only composed of a single noble metal and do not have a core-shell structure.
